(19) United States
(12) Patent Application Publication
(10) Pub. No.: US 2012/0123263 A1
(43) Pub. Date: May 17, 2012

(54) ULTRASONIC DIAGNOSTIC APPARATUS AND ULTRASONIC MEASUREMENT METHOD

(75) Inventors: Takashi Osaka, Tokyo (JP); Tsuyoshi Mitake, Tokyo (JP); Akiko Tonomura, Tokyo (JP)

(73) Assignee: HITACHI MEDICAL CORPORATION, Chiyoda-ku, Tokyo (JP)

(21) Appl. No.: 13/382,630

(22) PCT Filed: Jul. 7, 2010
(86) PCT No.: PCT/JP2010/059242

§ 371 Date: Jan. 6, 2012

(30) Foreign Application Priority Data
Jul. 7, 2009 (JP) 2009-160536

(51) Int. Cl. A61B 8/14

(52) U.S. Cl. 600/438

(57) ABSTRACT

Disclosed are an ultrasonic diagnostic apparatus and an ultrasonic measurement method enabling easy acquisition of elasticity information by means of a shear wave.

The ultrasonic diagnostic apparatus is provided with an ultrasonic probe (4) configured to transmit/receive an ultrasonic wave to/from an object (5), a vibrator (3) configured to generate a shear wave, a transmission/reception units (2,6) configured to transmit/receive an ultrasonic wave to/from the ultrasonic probe (4), a shear wave propagation detecting unit (14) for determining the propagation position of the shear wave and the propagation time of the shear wave, a shear wave image constructing unit (16) configured to construct a shear wave image showing the relation between the propagation position and propagation time of the shear wave, and an elasticity information calculating unit (15) configured to calculate elasticity information based on the boundary of the shear wave image.

### FIELD OF THE INVENTION

The present invention relates to an ultrasonic diagnostic apparatus and ultrasonic measurement method for acquiring and displaying elasticity information on biological tissue, etc. using ultrasonic waves.

### DESCRIPTION OF RELATED ART

An ultrasonic diagnostic apparatus transmits an ultrasonic wave to biological tissue, etc. via an ultrasonic probe, receives the reflected echo signal of the ultrasonic wave according to the structure of biological tissue, etc., generates and displays an ultrasonic tomographic image. Also, the ultrasonic diagnostic apparatus is capable of pressing biological tissue, etc. via an ultrasonic probe manually or mechanically, acquiring displacement of the biological tissue based on the frame data of the two ultrasonic waves measured at different times, and generating an elastic image showing elasticity information on softness or hardness of biological tissue from the acquired displacement data.

Further, there is a method for acquiring elasticity information using ultrasonic waves by using the wave referred to as a shear wave produced by low-frequency vibration (about ˜1 kHz) added to biological tissue, etc. Propagation velocity of a shear wave indicates hardness of a propagation medium and is proportional to the square root of shear elasticity coefficient, thus elasticity information on a biological tissue can be acquired by measuring the propagation velocity of a shear wave by an ultrasonic wave. Such a technique is disclosed in Patent Documents 1 and 2.

### PRIOR ART DOCUMENTS

Patent Document 1: JP-T-2005-534455
Patent Document 2: JP-A-2007-44231

Though an exclusive probe is used for measuring propagation velocity of a shear wave in Patent Document 1, since this exclusive probe is not capable of obtaining a tomographic image of an object, propagation velocity of a shear wave for acquiring elasticity information cannot be measured while observing a tomographic image.

Also, while mounting of an exclusive probe for measuring propagation velocity of a shear wave to a probe for obtaining a tomographic image is disclosed in Patent Documents 1 and 2, having two probes and two ultrasonic transmission/reception units for obtaining a tomographic image and for measuring propagation velocity of a shear wave makes operation and configuration thereof complicated.

The objective of the present invention is to provide the ultrasonic diagnostic apparatus and the ultrasonic measurement method capable of easily acquiring elasticity information by a shear wave.

### BRIEF SUMMARY OF THE INVENTION

In order to solve the above-described problems, the ultrasonic diagnostic apparatus of the present invention comprises:

an ultrasonic probe configured to transmit/receive ultrasonic waves to/from an object to be examined;

a vibrator configured to cause the object to generate a shear wave;

a transmission/reception unit configured to transmit/receive the ultrasonic waves between the ultrasonic probe and the ultrasonic diagnostic apparatus;

a shear wave propagation detecting unit configured to acquire propagation position of the shear wave and propagation time of the shear wave;

a shear wave image constructing unit configured to construct a shear wave image showing the relationship between the propagation position and the propagation time of the shear wave; and

an elasticity information calculating unit configured to calculate elasticity information based on image information of the shear wave image.

A shear wave image showing the relationship between the propagation position and the propagation time of a shear wave acquired by transmission/reception of an ultrasonic wave varies depending on the hardness of biological tissue which is the propagation medium of a shear wave. Normally, a shear wave image is represented by a line including a straight line and/or a curved line which contains slight errors caused at the time of detecting propagation position of a shear wave. However, there is often a considerable divergence in propagation position between a certain point on the line and the other points, which is attributed to an error or noise. Generally hardness of biological tissue should not vary greatly only at a certain point, thus erroneous elasticity information will be acquired by calculating propagation velocity of a shear wave based on such image.

In order to avoid such an error, the present invention calculates elasticity information based on a boundary of a shear wave image. In this manner, the certain point having a divergence in propagation position will be eliminated, whereby reducing the influence of an error or noise. The boundary of a shear wave image is the line including the straight line and/or the curved line which appear when the influence attributed to an error or noise is reduced.

Reduction of the influence attributed to an error or noise can be achieved by determining the boundary of a shear wave image on the basis of at least two points set on the shear wave image. By doing so, approximation is executed by a straight line between the two points to eliminate the point having a divergence in propagation position, the influence attributed to an error or noise can be reduced. As for the method of setting points, it is preferable to set them on the boundary of biological tissue based on the tomographic image to be described later, and the greater the number of points that are set the higher the accuracy that can be achieved. It also is preferable to provide an image preparation unit configured to execute image processing for improving quality of a shear wave image at the time of image construction.

As for elasticity information, propagation velocity or the Young's modulus can be used.

In this case, the elasticity information calculating unit can be configured to calculate elasticity information by automatically approximating the boundary of a shear wave image with a straight line. In this manner, the shear wave image in which the boundary is automatically approximated with a straight line can be constructed by setting a range on the shear wave image. At this time, the method for setting the points should be determined in advance.

The transmission/reception unit is configured to transmit an ultrasonic wave for detecting propagation position of a shear wave from at least one aperture set in the ultrasonic probe in the setting direction of an object and execute reception process on the reflected echo signal of the transmitted ultrasonic wave for detecting propagation position of a shear wave, so as to generate a plurality of RF line signals. Propagation position of a shear wave can be obtained by the time that the ultrasonic wave for detecting propagation position reflects to a shear wave and returns, and the velocity of the ultrasonic wave.

In this case, the image constructing unit can also be configured to construct a tomographic image based on the reflected echo signal received and processed by the transmission/reception unit.

In this way, scanning can be performed while transmitting and receiving the ultrasonic wave for obtaining a tomographic image using the same ultrasonic probe, and propagation position of a shear wave can be detected while obtaining a tomographic image by resting transmission/reception of the ultrasonic wave for obtaining a tomographic image at every pre-set time interval and transmitting/receiving the ultrasonic wave for detecting propagation position of a shear wave in the setting direction during the rest state. Thus a tomographic image and the elasticity information by the shear wave can be acquired without having two probes for obtaining a tomographic image and for measuring propagation velocity. As a result, the relationship between the propagation position of the shear wave and the propagation time of the shear wave can be acquired, whereby the propagation velocity of the shear wave can be calculated.

Also, the image construction unit can be configured so that the shear wave image corresponding to the region selected on a tomographic image displayed on the image display unit can be displayed on the image display unit. In this manner, propagation velocity distribution can be easily recognized on the tomographic image, which improves availability in acquisition of elasticity information.

In this case, the image construction unit can also be configured to display a measurement line corresponding to the transmission line of the ultrasonic wave for detecting propagation position of a shear wave on a tomographic image which is displayed on the image display unit. In this manner, the line for acquiring elasticity information can be determined referring to the tomographic image. In the case of moving the line, the ultrasonic probe can be moved or the aperture for transmitting the ultrasonic wave for detecting propagation position of shear wave can be changed.

Further, the elasticity information calculating unit that calculates elasticity modulus distribution in the setting direction based on a boundary can be provided in the ultrasonic diagnostic apparatus. Also, the image construction unit can be configured to display elasticity modulus distribution so that the elasticity modulus distribution is corresponded to and synthesized with the tomographic image displayed on the image display unit. In this manner, propagation modulus distribution can be easily recognized on the tomographic image which improves availability in acquisition of elasticity information.

Also, the image construction unit can be configured to construct an M-mode image based on the reflected echo signal received and processed by the transmission/reception unit, and to juxtapose and display the tomographic image and the shear wave image on the image display unit on one screen. Further, the transmission/reception unit can also be configured to detect change on an M-mode image and to start transmission of an ultrasonic wave for detecting propagation position of a shear wave.

Also, transmission/reception unit can be configured to generate RF frame data by receiving and processing the reflected echo signal in the process of adding pressure to an object via the ultrasonic probe, and the image construction unit can be configured to construct an elastic image from the RF frame data and display the elastic image by replacing it with the tomographic image.

Also, the ultrasonic-wave measuring method of the present invention, in the process of receiving an ultrasonic wave by an ultrasonic probe that transmits/receives the ultrasonic wave, causes a vibrator to make an object to generate a low-frequency shear wave, transmits/receives the ultrasonic wave for detecting the propagation position of the shear wave in the setting direction of the object, acquires the propagation position and the propagation time of the shear wave on the setting direction, generates and displays a shear wave image showing the relationship between the propagation position and the propagation time of the shear wave, and calculate elasticity information based on the boundary of the shear wave image.

### Effect of the Invention

In accordance with the present invention, it is possible to provide the ultrasonic diagnostic apparatus capable of easily acquiring elasticity information by shear waves.

### BRIEF DESCRIPTION OF THE DIAGRAMS

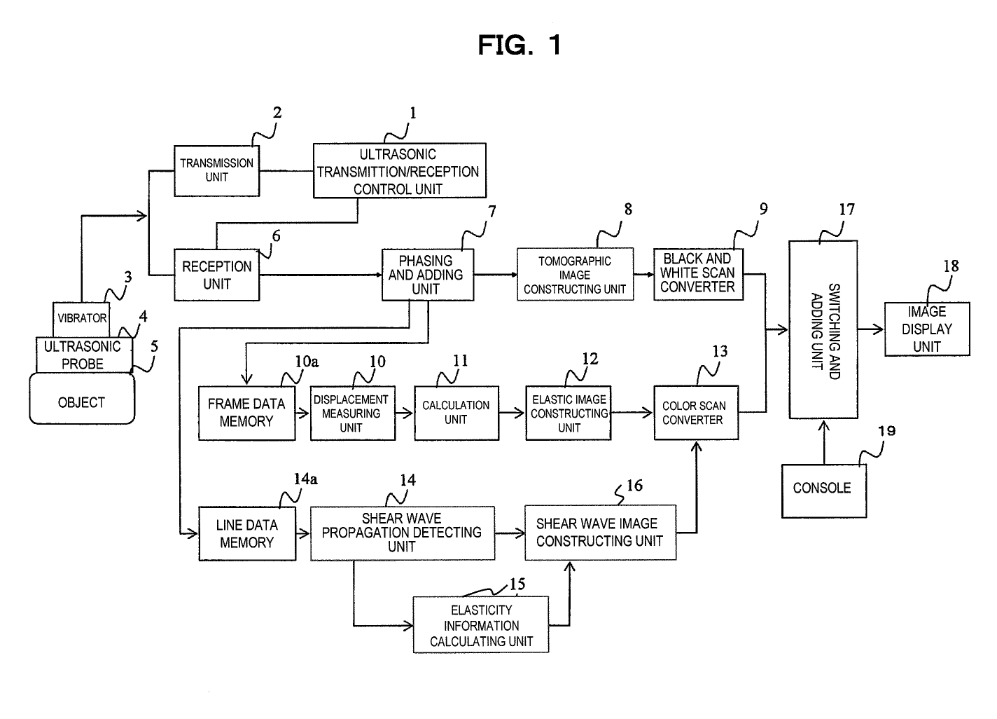

FIG. 1 is a block diagram of the ultrasonic diagnostic apparatus related to the present invention.

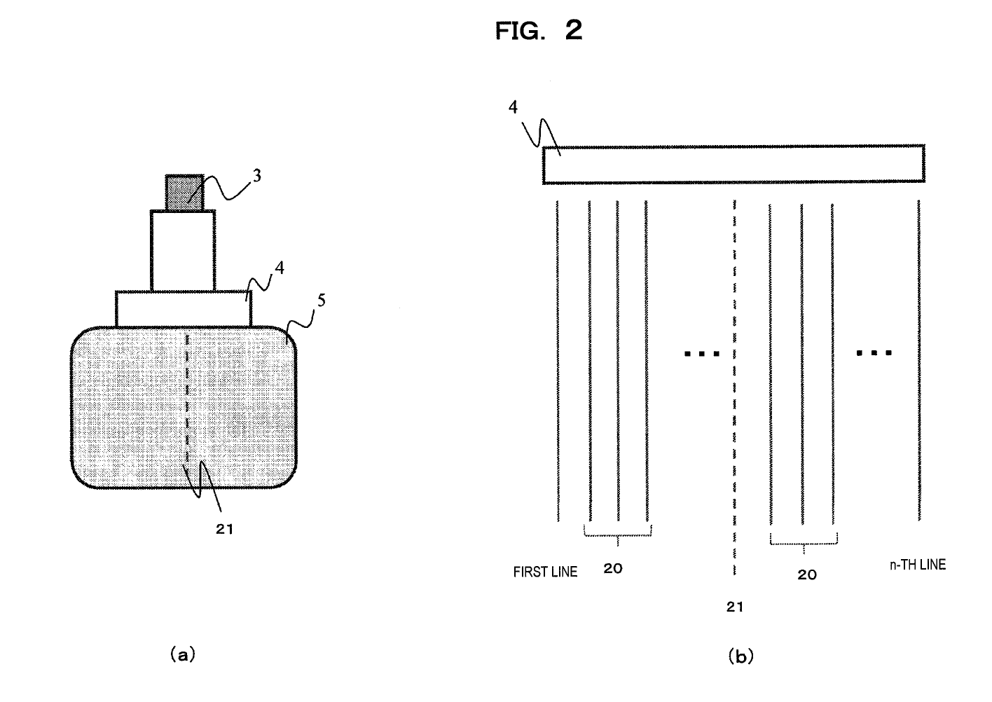

FIG. 2(a) is a view showing a configuration of an ultrasonic probe, and FIG. 2(b) is a view showing an ultrasonic wave transmitted from an ultrasonic probe.

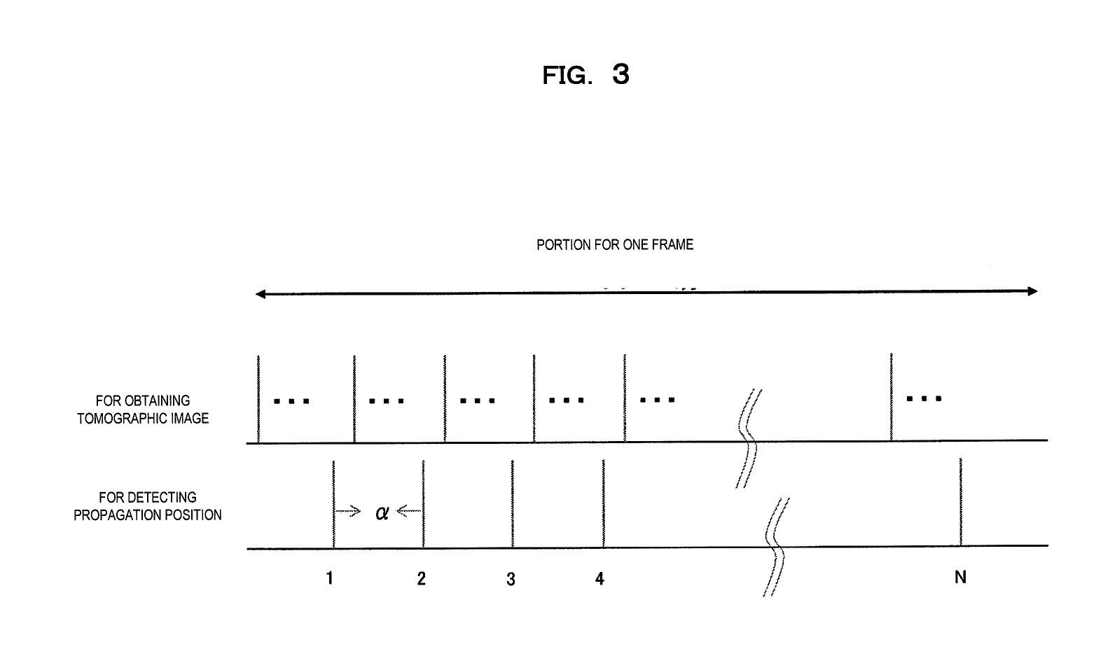

FIG. 3 is a timing chart of ultrasonic-wave transmission and reception.

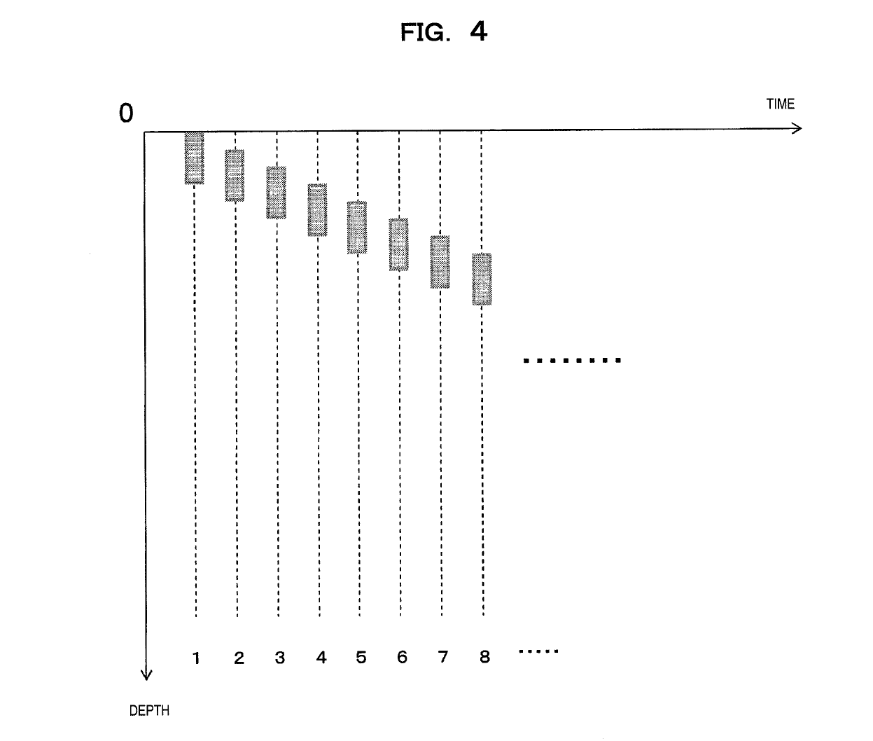

FIG. 4 is a view showing the relationship between the depth and the time of a shear wave.

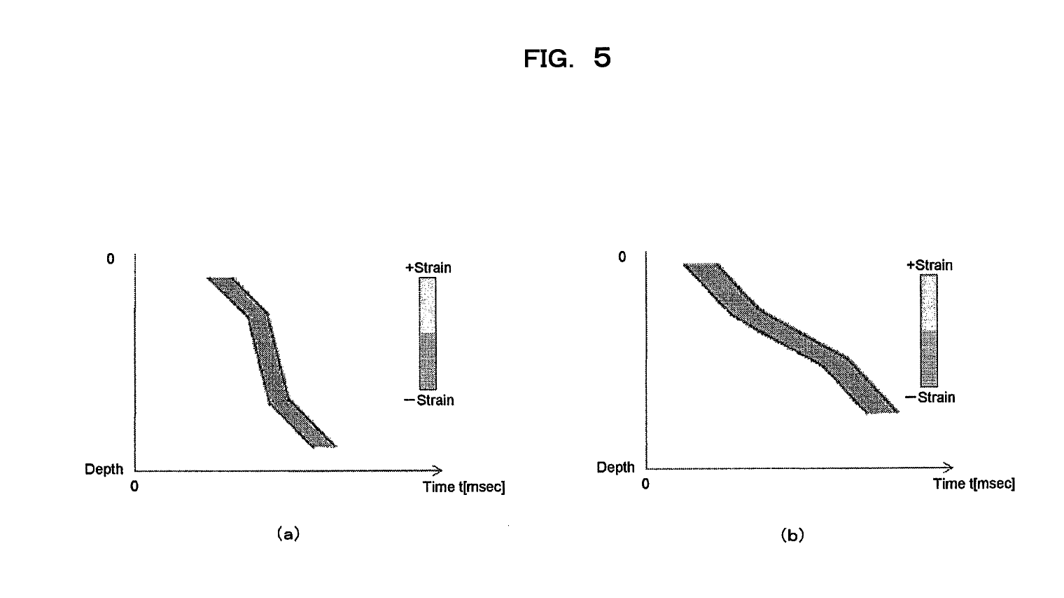

FIG. 5 is an example of images showing propagation velocity distribution displayed on an image display unit.

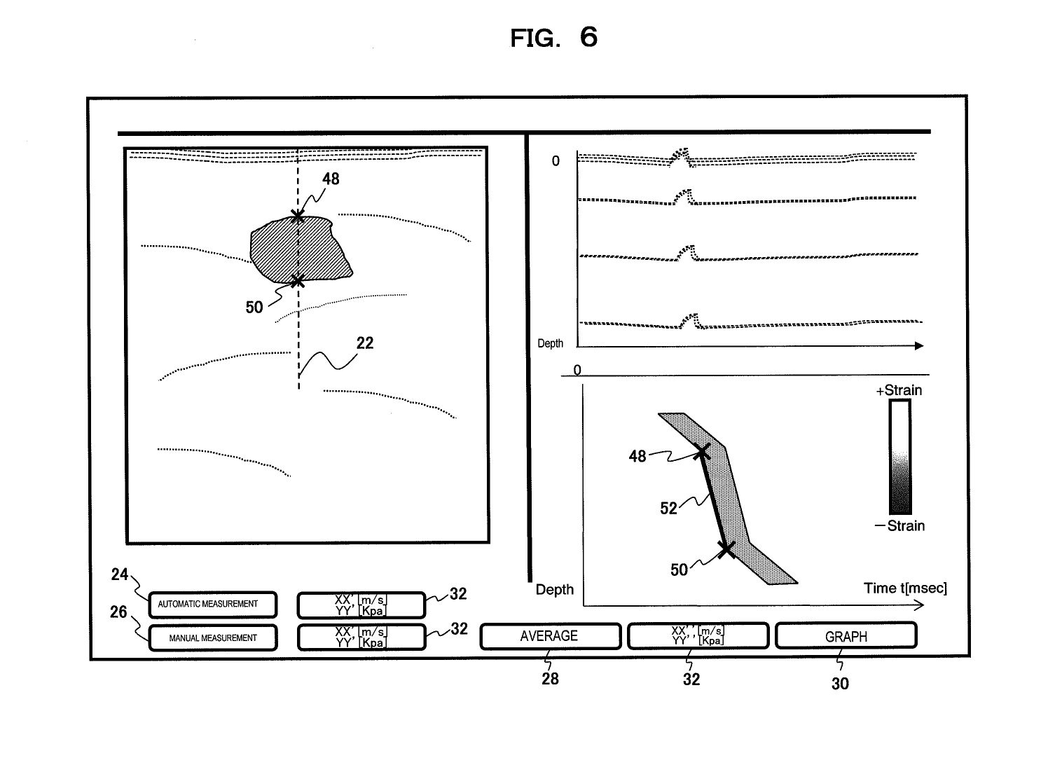

FIG. 6 is a view showing a screen on which a B-mode image, an M-mode image and a propagation velocity distribution image are displayed.

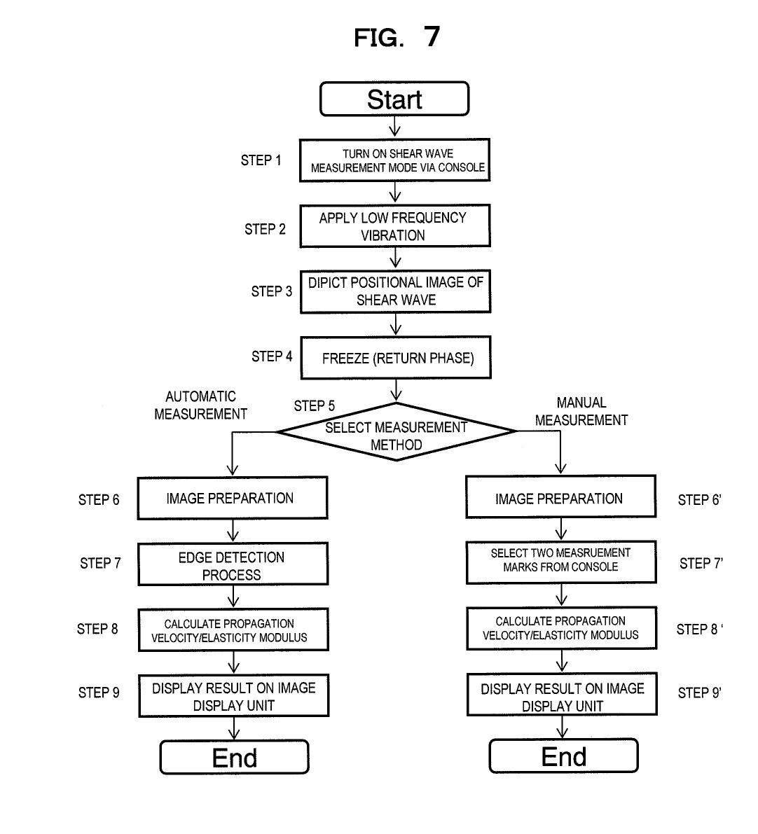

FIG. 7 is a flowchart for edge detection.

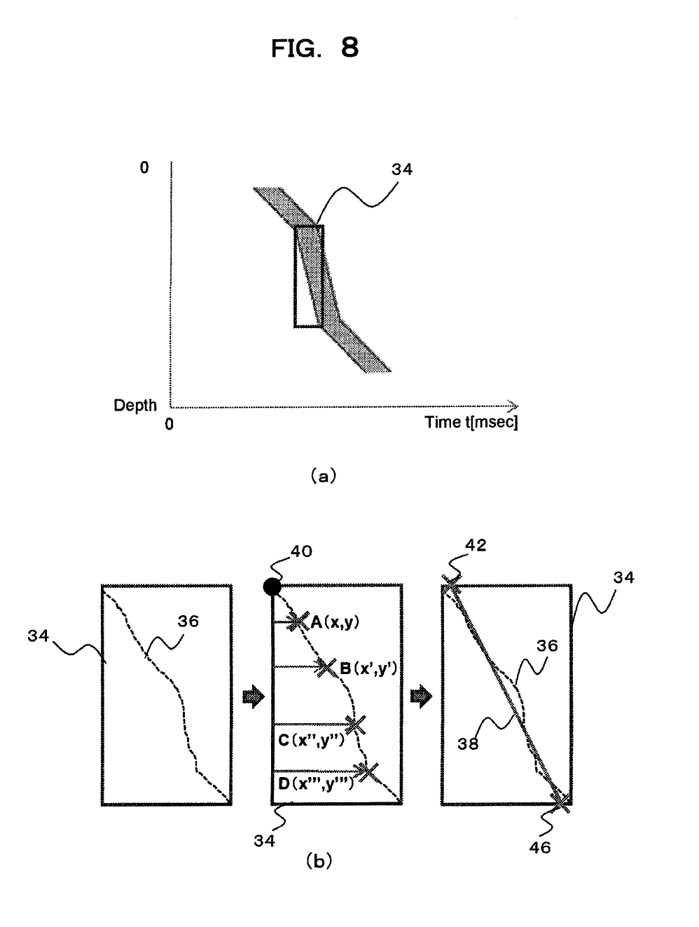

FIGS. 8(a) and (b) are views showing procedure of edge detection in automatic measurement.

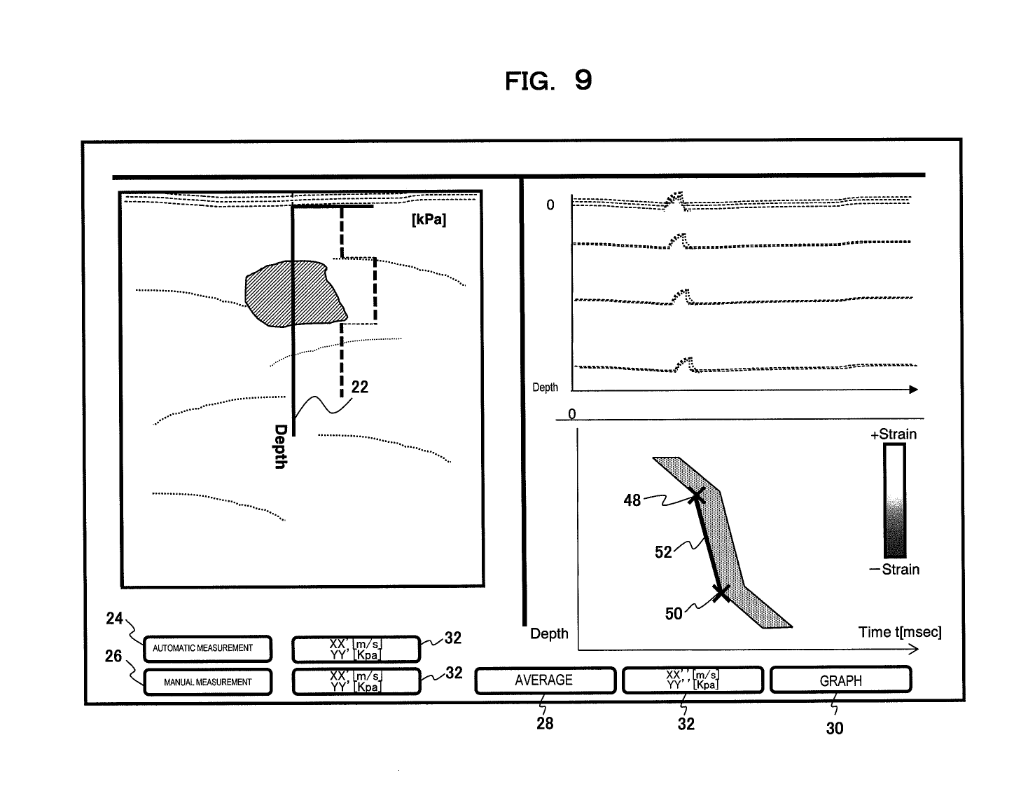

FIG. 9 is a view showing a screen on which a graph of elasticity information on a B-mode image is displayed.

### DETAILED DESCRIPTION OF THE INVENTION

The preferable embodiment of the ultrasonic diagnostic apparatus related to the present invention will be described below referring to the attached diagrams. As shown in FIG. 1, the ultrasonic diagnostic apparatus of the present embodiment comprises an ultrasonic probe 4 configured to transmit/receive an ultrasonic wave to/from an object 5, a vibrator 3 capable of being attached/detached to/from the ultrasonic probe 4 configured to apply a low-frequency vibration to the object 5 via the ultrasonic probe 4 to generate a shear wave, a transmission unit 2 configured to repeatedly transmit an ultrasonic wave to the object 5 via the ultrasonic probe 4 at time intervals, a reception unit 6 configured to receive the reflected echo signal produced from the object 5 in time series, an ultrasonic transmission/reception control unit 1 configured to control the transmission unit 2 and the reception unit 6, and a phasing and adding unit 7 configured to perform phasing and adding of the reflected echo signals received by the reception unit 6.

Also, the ultrasonic diagnostic apparatus of the present embodiment comprises a tomographic image constructing unit 8 configured to construct a grayscale image, for example a black and white tomographic image of the object 5 based on the RF (Radio Frequency) frame signal from the phasing and adding unit 7, and a black and white scan converter 9 configured to convert the output signal of the tomographic image constructing unit 8 to match the display of the image display unit 18.

Also, the ultrasonic diagnostic apparatus of the present embodiment comprises a frame data memory 10a configured to store the RF frame signals outputted from the phasing and adding unit 7, a displacement measurement unit 10 configured to measure the displacement generated in biological tissue of the object 5, a calculation unit 11 configured to obtain strain or elasticity modulus for calculating elasticity information in continuous pressing procedure from the displacement information measured by the displacement measurement unit 10, an elastic image constructing unit 12 configured to construct a color elastic image from the strain or the elasticity modulus calculated by the calculation unit 11, and a color scan converter 13 configured to convert the output signals from the elastic image constructing unit 12 to match the display of the image display unit 18.

Here, the characteristic configuration of the present embodiment will be described. The ultrasonic diagnostic apparatus further comprises a line data memory 14a configured to store the RF line signal to be described later in detail outputted from the phasing and adding unit 7, a shear wave propagation detecting unit 14 configured to acquire the propagation position and the propagation time of a shear wave, an elasticity information calculating unit 15 configured to obtain strain information for calculating elasticity information from propagation velocity, and a shear wave image constructing unit 16 configured to generate a shear wave image on the basis of the time axis from the output of the shear wave image propagation detecting unit 14, wherein the output signal of the shear wave image constructing unit 16 is converted to match the display of the image display unit 16 by the color scan converter 13.

The ultrasonic diagnostic apparatus further comprises an adding unit 17 configured to superimpose, juxtapose or switch a black and white tomographic image and a color elastic image, an image display unit 18 configured to display synthesized images, and a console 19 configured to select and operate images.

Here, operation of general components in the ultrasonic diagnostic apparatus related to the present embodiment will be described. The ultrasonic probe 4 is configured by disposing a plurality of transducers, to transmit/receive ultrasonic waves via the transducers to/from the object 5. The transmission unit 2 generates a transmission pulse for producing an ultrasonic wave by driving the ultrasonic probe 4, and sets the convergent point of the transmitted ultrasonic waves at a certain depth.

Also, the reception unit 6 amplifies the reflected echo signal received by the ultrasonic probe 4 at a predetermined gain, and generates an RF signal, i.e. a wave-receiving signal. The phasing and adding unit 7 inputs and controls the phase of the RF signal amplified by the reception unit 6, and generates an RF frame signal by forming an ultrasonic beam with respect to one or more convergent points. The tomographic image constructing unit 8 executes signal processing such as gain compensation, log compression, detection, edge enhancement and filtering by inputting the RF frame signal from the phasing and adding unit 7 so as to obtain tomographic image data of an image such as a B-mode image or an M-mode image.

The black and white scan converter 9 is configured including an analogue/digital converter (now shown) configured to convert the tomographic image data from the tomographic image constructing unit 8 into digital signals, a frame memory configured to store plural sets of converted tomographic image data in time series, and a controller. The black and white scan converter 9 obtains the tomographic frame data in the object 5 stored in the frame memory as one image, and reads out the obtained tomographic frame data with TV synchronism.

Next, operation for the case of obtaining elasticity information by pressing the object 5 via the ultrasonic probe 4 will be described. The RF frame signal outputted from the phasing and adding unit 7 is arbitrarily selected and recorded in the frame data memory 10a. The displacement measurement unit 10 acquires one-dimensional or two-dimensional displacement distribution related to the displacement or moving vector, i.e. the direction and the size of displacement in the biological tissue corresponding to the respective points of a tomographic image from a set of data in the frame data memory 10a by executing a one-dimensional or two-dimensional correlation process.

An example of the method for detecting a moving vector is the block matching method. The block matching method divides an image into blocks formed by, for example N×N pixels, focuses on the block within the region of interest, searches the block which is most approximated to the focused block from the previous frame, and determines the sample value by the predictive coding, i.e. the difference referring to the approximated block.

The calculation unit 11 calculates strain or elasticity modulus with respect to the data outputted from the displacement measurement unit 10. For example, when calculating elasticity modulus, the pressure value measured by a pressure sensor (not shown) connected to the ultrasonic probe 4 can be used, and the strain data needs to be calculated from the output data of the displacement measurement unit 10. The strain data can be calculated by executing spatial differentiation on the amount of movement, for example the displacement of biological tissue. Also, elasticity modulus data can be calculated by dividing the change of pressure by the change of strain.

By setting the displacement measured by the displacement measurement unit 10 as L(x) and the pressure measured by the pressure sensor as P(x), strain ΔS(x) can be calculated by executing spatial differentiation on L(x), thus by using the equation of ΔS(x)=ΔL(x)/Δx.

Also, Young's modulus Ym(x) which is elasticity information can be calculated by the equation of Ym(x)=(ΔP(x))/S(x). The elasticity modulus of the biological tissue equivalent to the respective points of a tomographic image can be acquired by the Young's modulus, thus the 2-dimensional elastic image data can be continuously obtained. The Young's modulus is the ratio between the simple tensile stress added to an object and the strain generated parallel to the tensile.

The elastic image constructing unit 12 is formed by a frame memory and an image processing unit (not shown), configured to store the elasticity frame data outputted from the calculation unit 11 in time series in the frame memory and executes a desired image processing to the stored frame data. The color scan converter 13 gives hue information to the elastic image data from the elastic image constructing unit 12 and a shear wave image constructing unit 16 to be described later.

In other words, the color scan converter 13 converts image data into the lights three primary colors, i.e. red (R), green (G) and blue (B) based elasticity frame data. For example, the elasticity data having large strain is converted to read-color code, and the elasticity data having small strain is converted to blue-color code.

Here, operation of the characterized configuration in the present embodiment will be described. As described above, it is necessary to apply a low-frequency vibration of about ˜1 kHz in order to cause the object 5 to generate a shear wave. Therefore, a detachable vibrator 3 is attached to the ultrasonic probe 4 as shown in FIG. 2(a). The vibration to be generated from the vibrator 3 may either be continuous or a single-shot. Propagation velocity of an ultrasonic wave in a human body is about 1530 m/second, and propagation velocity of a shear wave is about 1˜5 m/second.

The ultrasonic waves to be irradiated to the object at this time from the transmission unit 2 via the ultrasonic probe 4 are an ultrasonic wave 20 for obtaining a tomographic image and an ultrasonic wave 21 for detecting propagation position of a shear wave as shown in FIG. 2(b). The ultrasonic wave 20 for obtaining a tomographic image is transmitted by sequentially switching the transducers that are arrayed in the ultrasonic probe 4. The transmitting direction of the ultrasonic wave 21 for detecting propagation position of a shear wave is to be determined in advance. It is the depth direction of the object 5 in the present embodiment.

Also, the ultrasonic wave 21 for detecting propagation position is transmitted only from the transducer preset as a channel from among the plurality of transducers arrayed in the ultrasonic probe 4. In FIG. 2(b), the transducer disposed in the middle of the ultrasonic probe 4 is set as a channel. FIG. 3 is a transmission timing chart of the ultrasonic wave 20 for obtaining a tomographic image and the ultrasonic wave 21 for detecting propagation position. As shown in FIG. 3, the ultrasonic wave 21 for detecting propagation position is transmitted one time for each time that a plurality of ultrasonic waves 20 for detecting a tomographic image are transmitted, and the transmission interval is α. α is a PRF (pulse-repeating frequency) of the ultrasonic wave 21 for detecting propagation position, and is transmitted plural times in one frame.

Such transmitted reception signals of the ultrasonic waves 21 for detecting propagation position are sequentially recorded in the line data memory 14a. The shear wave propagation detecting unit 14 acquires the relationship between the depth and the propagation time of the shear wave. This reception signal is the ultrasonic wave 21 for detecting propagation position which is influenced upon hitting against and reflecting a shear wave. The shear wave propagation position detecting unit 14 acquires propagation information of the shear wave from a plurality of reception signals. Propagation information of a shear wave includes propagation position and propagation time of the shear wave. Propagation position of a shear wave can be acquired by the time that the ultrasonic wave 21 for detecting propagation position reflects on a shear wave and returns, and by the velocity of an ultrasonic wave.

FIG. 4 is a graph showing the relationship between the depth of a shear wave (vertical axis) which indicates the position thereof and the time (lateral axis). The rectangles in FIG. 4 indicate displacements generated along with propagation of a shear wave, wherein the width of the rectangle in the vertical direction in the diagram is equivalent to the wavenumber of a shear wave and the width in the lateral direction in the diagram is equivalent to the amplitude. The shear wave is propagated inside of the object 5 with time, and the propagation velocity can be calculated using the depth and the time (inverse number of α) acquired by the ultrasonic wave 21 for detecting propagation position.

The elasticity information calculating unit 15 calculates elasticity information by a shear wave from its propagation velocity. When the Young's modulus is set as E, density of a medium is set as p and the propagation velocity is set as Vs, they can be expressed as the equation of E=3ρVs2, and the Young's modulus can be calculated using the equation.

The shear wave image constructing unit 16 constructs a shear wave image showing the relationship between the depth and the propagation time of the shear wave acquired by the shear wave propagation detecting unit 14 and a graph of elasticity information by the shear wave acquired by the elasticity information calculating unit 15, and the color scan converter 13 generates an image thereof. FIGS. 5(a) and (b) are examples of a shear wave image. In FIGS. 5(a) and (b), the longitudinal axis indicates the depth (the upper part is 0), the lateral axis indicates the time, and the gradient shows the propagation velocity. The faster the propagation velocity is the harder the medium is, thus the figures indicate that the media in FIG. 5(a) is harder than the media in FIG. 5(b). The Young's modulus is indicated by the longitudinal axis and the depth is indicated by the lateral axis in the graph showing elasticity information by a shear wave, and is shown on a B-mode image of FIG. 9 in the present embodiment.

The switching and adding unit 17 comprises a frame memory, image processing means and image selecting means (not shown). Here, the frame memory stores tomographic image data from the black and white scan converter 9 and elastic image data from the color scan converter 13, and the image processing means synthesizes the tomographic image data and the elastic image data (including a shear wave image) by changing the proportion of synthesis.

Brightness information and hue information of the respective pixels in the composite image is the addition of the respective information in the black and white tomographic image and the color elastic image by the proportion of synthesis. Further, the image to be displayed on the image display unit 18 is selected from among the tomographic image data and the elastic image data in the frame memory and the synthetic image data in the image processing unit by the image selecting means.

FIG. 6 is a view showing an example of a screen to be displayed on the image display unit 18. In FIG. 6, three kinds of images are depicted which are a B-mode image, an M-mode image and a shear wave image. A B-mode image is displayed in real time on the left part of FIG. 6, and a measurement line 22 indicating the position for acquiring elasticity information by a shear wave is displayed on this B-mode image.

The buttons for an automatic measurement 24, a manual measurement 26, an average 28, a graph 30, a result display 32, a starting point 48 and an ending point 50 shown on a display screen of FIG. 6 will be described later.

An examiner can freely change the position of the measurement line 22 using the console 19. By setting the measurement line 22, the channel for transmitting the ultrasonic wave 21 for detecting propagation position of a shear wave can be determined. An M-mode image of the measurement line 22 is depicted on the upper-right part of FIG. 6 and a shear wave image of the measurement line 22 is depicted on the lower-right part of FIG. 6 by translucent display.

The examiner executes an examination while confirming the B-mode image depicted on the left side of FIG. 6, and determines the cross section for acquiring a shear wave image. The measurement line 22 is set with respect to the determined cross section (set in the center portion in FIG. 6). In this condition, the examiner manually operates the vibrator 3 attached to the ultrasonic probe 4, causes the object 5 to generate a shear wave by applying a low-frequency vibration, and generates a shear wave image as previously described.

The ultrasonic diagnostic apparatus of the present embodiment comprises the ultrasonic probe 4 configured to transmit/receive an ultrasonic wave to/from the object 5, the vibrator 3 configured to cause the object 5 to generate a shear wave, the transmission/reception units 2 and 6 configured to execute transmission/reception of an ultrasonic wave to/from the ultrasonic probe 4, the shear wave propagation detecting unit 14 configured to acquire propagation position and propagation time of a shear wave, the shear wave image constructing unit 16 configured to construct a shear wave image showing the relationship between propagation position and propagation time of a shear wave, and elasticity information calculating unit 15 configured to calculate elasticity information based on the image information of a shear wave image. Image information of a shear wave image includes the boundary (edge) of a shear wave image, the shape of a shear wave image, and the relationship between propagation position and propagation time of a shear wave. Elasticity information includes propagation velocity of a shear wave, the Young's modulus, and so on.

As previously described, propagation velocity and elasticity information of a shear wave can be acquired by generating a shear wave image. Consequently, improving detection accuracy of the boundary (edge) of a shear wave image leads to the improvement in accuracy of propagation velocity and elasticity information. Given this factor, edge detection will be described. The edge here includes a straight line and/or a curved line that appears when the influence attributed to an error or noise in a shear wave image is reduced, and a straight line is exemplified in the present embodiment.

FIG. 7 is a flowchart for edge detection. Edge detection is to be executed by automatic or manual extracting operation. Here, step 1˜step 5 in FIG. 7 that are common in automatic and manual operation will be described. In addition, while shear wave images in FIGS. 5, 6 and 9 are depicted by straight lines for illustrative purposes, actual images are not straight lines due to error or noise, and are depicted by straight lines after edge detection.

The examiner switches from a normal screen for ultrasonic diagnosis (B-mode image) to the condition capable of acquiring elasticity information by a shear wave as shown in FIG. 6 by operating the console 19 (step 1). The examiner executes ultrasonic diagnosis using a B-mode image and determines the cross section for acquiring elasticity information by a shear wave using the measurement line 22 at the same time (step 2).

After determining the cross section, the examiner applies a vibration to the object 5 by operating the vibrator 3 which is attached to the ultrasonic probe 4 (step 3). When a shear wave image is depicted by the vibration from the vibrator 3, the examiner sets the ultrasonic diagnostic apparatus in a freeze-up state (step 4). The examiner selects one measurement method via the console 19 from either the automatic measurement 24 or the manual measurement 26 that are displayed on the image display unit 18 (step 5).

The automatic measurement 24 automatically acquires the boundary (edge) of a shear wave image for acquiring elasticity information, and the manual measurement 26 is operated manually by the examiner to acquire the boundary of a shear wave image.

After selecting the measurement method, image preprocessing is executed for improving accuracy of edge detection (step 6 and step 6′). An example of preprocessing is the commonly-known dilation/contraction processing which is referred to as morphology. By combining these processing or executing the processing plural times, the edge of velocity gradient can be sharpened.

The case of automatic measurement after step 7 will now be described referring to FIG. 8. When the examiner selects and holds down the button of the automatic measurement 24 in FIG. 6, an edge detection region 34 shown in FIG. 8(a) is displayed on the image display unit 18. FIG. 8(a) shows the case that a hard biological tissue is detected on the measurement line 22 shown in FIG. 6, and FIG. 8(b) shows the process for detecting a final edge 38 by executing the edge detection process on a shear wave image 36.

The examiner sets the edge detection region 34 on a shear wave image via the console 19. As shown in FIG. 9(b), after the edge detection region 34 is set, the leftmost-top position is set as a search-start reference point 40, and edge detection is executed toward the far right of the edge detection region. By executing this operation at several points (four points in the present embodiment) with respect to the depth direction at predetermined intervals, the respective coordinate values for edge coordinates A(x,y), B(x′,y′), C(x″,y″), and D(x′″,y′″) can be detected. On the basis of the detected coordinate values, the elasticity information calculating unit 15 calculates the line segments having the shortest distance from the respective coordinate points using the least-square method, and determines a final edge 38 which connects a final edge start-point 42 and a final edge end-point 46 (step 7).

By calculating the final edge 38 in step 7, propagation time T and propagation distance (depth) D of the shear wave are determined. The elasticity information calculating unit 15 calculates propagation velocity Vs of the shear wave using the propagation time T and the propagation distance D, and calculates the Young's modulus using the previously mentioned formula (step 8). While the boundary (edge) of a shear wave image is used above as the image information, the shape of a shear wave image (gradient, etc. of a shear wave image) may also be used to determine propagation time T and propagation distance (depth) D of a shear wave.

Next, the case of manual measurement after step 7′ will be described. When the examiner holds down and selects the button of the manual measurement 26 in FIG. 6, a start point 48 and an end point 50 for determining the straight line along a shear wave image as shown in FIG. 6 are sequentially displayed. The examiner determines both the start point 48 and the end point 50 along the shear wave image. A segment line 52 which connects the start point 48 and the end point 50 becomes the straight line which indicates the propagation velocity. Also as shown in FIG. 6, the start point 48 and the end point 50 can be also determined from the corresponding B-mode image (step 7′).

As with automatic measurement, propagation time T and propagation distance D are determined with respect to the line segment 52 calculated in step 7′, and the propagation velocity and the Young's modulus (elasticity information) of the shear wave are calculated (step 8′).

The propagation velocity and the Young's modulus of a shear wave calculated in step 8 and step 8′ of the flowchart in FIG. 7 are respectively displayed on a result display 32 shown in step 9 and step 9′ of FIGS. 6 and 9. In this manner, the examiner can recognize the measured elasticity information as quantitative numeric values.

Also by holding down and selecting the button of the average button 28 in FIGS. 6 and 9, the examiner can calculate the average value between the propagation velocity and the Young's modulus calculated from the edge detected by automatic measurement and the propagation velocity and the Young's modulus calculated from the edge detected by manual measurement.

Further, by holding down the button of the graph 30 shown in FIGS. 6 and 9, elasticity information can be displayed in the form along the measurement line 22 as shown in FIG. 9. The vertical axis indicates the Young's modulus and the lateral axis indicates the depth in the displayed graph. By superimposing and displaying the graph over the measurement line 22, the elasticity information can be provided while being corresponded to the measurement line 22 which is determined by the examiner.

As described above, in accordance with the present embodiment, since the start point 48 and the end point 50 are set on a shear wave image so as to approximate between the two points by a straight line and the points of the propagation positions that are substantially away from the other interconnected parts on the line because of an error or noise are eliminated, it is possible to reduce the influence of the error or noise.

Also, since an ultrasonic wave 21 for detecting propagation position of a shear wave is transmitted one time for each time that plural ultrasonic waves 20 for obtaining a tomographic image are transmitted and scanned, the propagation position of the shear wave can be detected while obtaining the tomographic image. In this manner, it is possible to acquire elasticity information by a shear wave along with a tomographic image without having two probes, a probe for obtaining a tomographic image and a probe for measuring propagation velocity of a shear wave.

Also, it is possible to construct a shear wave image which is automatically approximated by a straight line, since a shear wave image is constructed by setting the edge detection region 34 on the image and automatically approximating by a straight line.

Also, since the measurement line 22 corresponding to a transmission line of the ultrasonic wave 21 for detecting shear wave propagation position is displayed on a B-mode image shown in FIG. 6, the line for acquiring elasticity information by a shear wave can be determined by referring to the B-mode image.

Also, the elasticity modulus distribution acquired by the elasticity information calculating unit 15 is displayed by being corresponded to and synthesized with a B-mode image, thereby improving means to confirm elasticity modulus distribution on a B-mode image.

The preferable embodiment of the present invention has been described. However, the present invention is not limited thereto and various kinds of alterations or modifications can be made. For example, the tomographic image constructed by the elastic image construction unit 12 may be displayed instead of a B-mode image in FIG. 6.

Also, while the ultrasonic wave 21 for detecting propagation of a shear wave is transmitted upon obtaining a B-mode image with or without generation of a shear wave, since a change occurs on an M-mode image in FIG. 6 when a shear wave is generated, it may be configured so that the ultrasonic wave 21 for detecting propagation position of a shear wave is transmitted when this change is detected.

It may also be configured that the image construction unit 16 displays the shear wave image corresponding to the region selected on a B-mode image of FIG. 6, thereby the shear wave image on a B-mode image can be easily recognized which improves usability of the apparatus.

### DESCRIPTION OF THE REFERENCE NUMERALS

2: transmission unit
3: vibrator
4: ultrasonic probe
6: reception unit
14: shear wave propagation detecting unit
14a: line data memory
15: elastic information calculating unit
16: shear wave image constructing unit
18: image display unit
20: ultrasonic wave for obtaining tomographic image
21: ultrasonic wave for detecting propagation position
22: measurement line
24: automatic measurement
26: manual measurement
34: region for edge detection
38: final edge

1. An ultrasonic diagnostic apparatus comprising:
an ultrasonic probe configured to transmit/receive an ultrasonic wave to/from an object to be examined;
a vibrator configured to cause the object to generate a shear wave;
a transmission/reception unit configured to transmit/receive the ultrasonic wave to/from the ultrasonic probe;
a shear wave propagation detecting unit configured to acquire propagation position and propagation time of the shear wave;
a shear wave image constructing unit configured to construct a shear wave image showing the relationship between propagation position and propagation time of the shear wave; and
an elasticity information calculating unit configured to calculate elasticity information based on image information of the shear wave image.

2. The ultrasonic diagnostic apparatus according to claim 1, wherein the elasticity information calculating unit calculates elasticity information based on at least two points being set on the shear wave image.

3. The ultrasonic diagnostic apparatus according to claim 1, wherein the elasticity information calculating unit calculates elasticity information by automatically approximating the boundary of the shear wave image with a straight line.

4. The ultrasonic diagnostic apparatus according to claim 1, wherein the elasticity information is the propagation velocity of a shear wave or the Young's modulus.

5. The ultrasonic diagnostic apparatus according to claim 1, wherein the transmission/reception unit transmits an ultrasonic wave for detecting propagation position of the shear wave in the set direction of the object from at least one aperture set in the ultrasonic probe and executes the reception process of the reflected echo signal of the transmitted ultrasonic wave for detecting propagation position of the shear wave, so as to generate a plurality of RF line signals.

6. The ultrasonic diagnostic apparatus according to claim 5, characterized in further comprising a tomographic image constructing unit configured to construct a tomographic image based on the reflected echo signal received and processed by the transmission/reception unit.

7. The ultrasonic diagnostic apparatus according to claim 6, wherein an image display unit displays the shear wave image corresponding to the region selected on the tomographic image.

8. The ultrasonic diagnostic apparatus according to claim 6, wherein an image display unit displays the measurement line corresponding to the transmission line of an ultrasonic wave for detecting propagation position of a shear wave on the tomographic image.

9. The ultrasonic diagnostic apparatus according to claim 6, wherein the elasticity information calculating unit calculates elasticity modulus distribution in the set direction based on a boundary.

10. The ultrasonic diagnostic apparatus according to claim 6, characterized in further comprising an image construction unit configured to construct an M-mode image based on the reflected echo signal received and processed by the transmission/reception unit, wherein the tomographic image, the shear wave image and the M-mode image are juxtaposed and displayed in one screen of an image display unit.

11. The ultrasonic diagnostic apparatus according to claim 6, characterized in further comprising an elastic image constructing unit configured to construct an elastic image from the ultrasonic wave, wherein an image display unit displays the elastic image.

12. The ultrasonic diagnostic apparatus according to claim 9, wherein an image display unit makes the elasticity modulus distribution and the tomographic image to be corresponded to and synthesized with each other to be displayed.

13. The ultrasonic diagnostic apparatus according to claim 10, wherein the transmission/reception unit detects a change on the M-mode image and starts transmitting the ultrasonic wave for detecting propagation position of a shear wave.

14. An ultrasonic measurement method, in the procedure of receiving an ultrasonic wave by an ultrasonic probe that transmits/receives ultrasonic waves to/from an object to be examined, which causes the object to generate a shear wave by a vibrator, acquires propagation position and propagation time of the shear wave, generates and displays a shear wave image showing the relationship between propagation position and propagation time of the shear wave, and calculates elasticity information based on the image information of the shear wave image.
